(19) United States
(12) Patent Application Publication
(10) Pub. No.: US 2017/0095410 A1
(43) Pub. Date: Apr. 6, 2017

(54) FOAMING COMPOSITION CONTAINING INTERNAL OLEFIN SULTONATES AND ONE FOAM-ENHANCING AGENT OR FOAM BOOSTER

(71) Applicant: L'OREAL, Paris (FR)

(72) Inventors: Ryuji HARA, Kawasaki-shi, Kawagawa (JP); Jinglan LI, Kawasaki-shi, Kawagawa (JP); Bradford PISTORIO, Kawasaki-shi, Kawagawa (JP)

(21) Appl. No.: 15/126,031

(22) PCT Filed: Mar. 11, 2015
(86) PCT No.: PCT/JP2015/057969

§ 371 Date: Sep. 14, 2016

(30) Foreign Application Priority Data
Mar. 19, 2014 (JP) 2014-056155

(51) Int. Cl. A61K 8/46; A61K 8/36; A61Q 5/02; A61K 8/34; B08B 3/08; A61K 8/73; A61K 8/88; A61K 8/39; B08B 3/00; A61K 8/04; A61K 8/19

(52) U.S. Cl. CPC A61K 8/466; A61K 8/046; A61K 8/361; A61Q 5/02; A61K 8/463; A61K 8/345; A61K 8/19; A61K 8/731; A61K 8/88; A61K 8/39; B08B 3/003; B08B 3/08; A61K 2800/49; A61K 2800/5426

(57) ABSTRACT

The present invention relates to a composition containing in an aqueous medium, at least 1) a sulfonate mixture containing: a) an internal olefin sulfonate (A) having 16 carbon atoms and b) an internal olefin sulfonate (B) having 18 carbon atoms,—wherein the weight ratio (A/B) of the component (A) to the component (B) present in the sulfonate mixture is from 75/25 to 90/10, preferably from 80/20 to 85/15, and—wherein the weight ratio of the hydroxy form compounds present in the internal olefin sulfonates (A) and (B) to the olefin form compounds present in the internal olefin sulfonates (A) and (B) is from 75/25 to 100/0, preferably from 80/20 to 95/5; and 2) at least one foam-enhancing agent or foam booster. The present invention relates also to a process for cleansing keratin materials, which consists of applying to the said keratin materials a composition according to the invention, in working the said composition into a foam and then rinsing off the said composition, especially with water. The present invention relates also to the cosmetic use of the composition as defined above, for removing makeup and/or cleansing the skin, the hair and/or mucous membranes, or for skincare.

The present invention relates to a composition containing in an aqueous medium, at least:
1) a specific sulfonate mixture which will be defined in greater detail hereinbelow and
2) at least one foam-enhancing agent or foam booster.

The present invention relates also to a process for cleansing keratin materials, which consists of applying to the said keratin materials a composition according to the invention, in working the said composition into a foam and then in rinsing off the said composition, especially with water.

The present invention relates also to the cosmetic use of the composition as defined above, for removing makeup and/or cleansing the skin, the hair and/or mucous membranes, or for skincare.

Cleansing the skin is very important for caring for the face. It must be as efficient as possible because greasy residues, such as excess sebum, the remnants of cosmetic products used daily, and make-up products, in particular waterproof products, accumulate in the skin folds, and can block the pores of the skin and result in the appearance of spots.

Several types of skin cleansing products, for example, rinsable cleansing anhydrous oils and gels, and foaming creams, lotions and gels, are known.

Rinsable anhydrous oils and gels have a cleansing action by virtue of oils present in these formulations. These oils make it possible to dissolve fatty residues and to disperse make-up pigments. These products are effective and well tolerated.

However, they exhibit the disadvantages of being heavy, of not foaming and of not conferring a feeling of freshness on application, which is disadvantageous from a cosmetic viewpoint.

On the other hand, foaming cleansing products have a cleansing action by virtue of the surfactants, which suspend the fatty residues and the pigments of, for example, the make-up products. They are effective and pleasant to use because they foam and because they are easy to remove. Visual as well as sensorial properties of foam are very important. Rapid formation of big volumes of a creamy foam is the important visual requirement, while a dense foam feel, often associated with creaminess, is the most wanted sensorial property.

Recently, cleansing products, in particular facial cleansing compositions, are expected to have not only cleansing effects and foaming ability but also to leave a good feeling on the skin after use, such as a fresh feeling, a not-tight feeling, a skin mildness and moisturizing sensation and a natural feeling.

Many foaming face cleansing products which are well-known in the prior art and are actually on the market are based on formulas that contain generally anionic surfactants of the sulphate type (ie alkylsulfates and alkylether sulfates). However, the drawback to these formulas is that, in order to provide a high level of foaming performance, they need to use high quantities of surfactants (>20%). Those high quantities of sulphate surfactants may lead to skin irritation and discomfort on the skin after use (tight feeling, dry sensation)—especially with long term use.

Thus, cleansing products which can contain other anionic surfactants than sulfates surfactant and have both good foamability performance (fast foam start, consistent foam, density, volume, and elasticity) and good feeling of the skin after use, are still desired.

Currently, on the market, sulfate-free personal care products in the body and hair care domains are proposed. However, few exist in the face wash category. In general, sulfate-free products are known to be gentle to the scalp and skin; and should therefore have a benefit in the face cleansing category, as there is an opportunity to provide face washes to consumers with sensitive skin. Nevertheless, a drawback to sulfate-free cleansers is that they tend to be low foaming—an important performance characteristic in the face cleansing domain particularly in Asia.

An objective of the present invention is to provide a composition, in particular a cleansing composition, which can contain anionic surfactants other than sulfate surfactants and which have good foamability performance and good feeling of the skin after use, such as fresh feeling, a not-tight feeling, skin mildness and moisturizing sensation and natural feeling; the said composition being free of the abovementioned drawbacks.

The inventors have demonstrated that this objective could be achieved with a composition, in particular a cleansing composition, containing in an aqueous medium:

1) at least a sulfonate mixture which will be defined in greater detail hereinbelow and

2) at least one foam-enhancing agent or foam booster.

One subject of the present invention is a composition, in particular a foaming cleansing composition, containing in a physiologically acceptable aqueous medium, at least:

1) a sulfonate mixture containing:

a) an internal olefin sulfonate (A) having 16 carbon atoms and

b) an internal olefin sulfonate (B) having 18 carbon atoms,

wherein the weight ratio (A/B) of the component (A) to the component (B) present in the sulfonate mixture is from 75/25 to 90/10, and

wherein the weight ratio of the hydroxy form compounds present in the internal olefin sulfonates (A) and (B) to the olefin form compounds present in the internal olefin sulfonates (A) and (B) is from 75/25 to 100/0; and

2) at least one foam-enhancing agent or foam booster.

Another subject of the present invention is a composition containing in an aqueous medium, at least:

1) a sulfonate mixture containing:

a) an internal olefin sulfonate (A1) having 16 carbon atoms which will be defined in greater detail hereinbelow and

b) an internal olefin sulfonate (B1) having 18 carbon atoms, which will be defined in greater detail hereinbelow and

optionally one or more compounds of formula (A2) and/or (A3) which will be defined in greater detail hereinbelow;

optionally one or more compounds of formula (B2) and/or (B3) which will be defined in greater detail hereinbelow;

wherein:

the weight ratio of all the compounds (A1), (A2) and (A3) to all the compounds (B1), (B2) and (B3) (which means (A1+A2+A3)/(B1+B2+B3)) is from 75/25 to 90/10; and

the weight ratio of all the compounds (A1) and (B1) to all the compounds (A2), (B2), (A3) and (B3) (which means (A1+B1)/(A2+A3+B2+B3)) is from 75/25 to 100/0; and

2) at least one foam-enhancing agent or foam booster.

Preferably, the said compositions contain a physiologically acceptable aqueous medium, and more particularly a cosmetically acceptable aqueous medium.

Contrary to all expectations, the inventors have found that the use of this cosmetic composition makes it possible to have both good foamability performance (fast foam start, consistent foam density, volume, and elasticity) without using necessarily sulphate surfactants and good feeling of the skin after use: fresh feeling, a not-tight feeling, skin mildness and moisturizing sensation and natural feeling.

According to another of its aspects, a subject of the invention is also a process for cleansing keratin materials, which consists in applying to the said keratin materials a composition according to the invention, in working the said composition into a foam and then in rinsing off the said composition, especially with water.

A subject of the present invention is also the cosmetic use of the composition as defined above, for removing makeup and/or cleansing the skin, the hair and/or mucous membranes, or for skincare.

For the purposes of the present invention, the term “physiologically acceptable medium” means a medium that is suitable for the topical administration of a composition. A physiologically acceptable medium is preferably a cosmetically or dermatologically acceptable medium, that is to say a medium which is devoid of unpleasant odour or appearance and which is entirely compatible with the topical administration route. Such a medium is in particular considered as physiologically acceptable when it does not cause the user any unacceptable stinging, tautness or redness.

For the purposes of the present invention, the term “foam-enhancing agent or foam booster” means any compound which improves foaming properties obtained by the cleansing surfactants like the foam start, foam speed, bubble size, foam volume, foam density and/or stability of the foam over time.

The compositions of the invention are preferably rinse-off compositions (rinsing with water or with a tonic) and they may be used in the field of the makeup removal and cleansing of facial or bodily skin, the hair, including the scalp, and mucous membranes such as the lips. They may also constitute care products, for instance rinse-off masks (in the usual manner in which these products are used).

Sulfonate Composition

The cosmetic composition according to the invention comprises a sulfonate mixture containing:

a) an internal olefin sulfonate (A) having 16 carbon atoms and

b) an internal olefin sulfonate (B) having 18 carbon atoms,

wherein the weight ratio (A/B) of the component (A) to the component (B) present in the sulfonate mixture is from 75/25 to 90/10, and

wherein the weight ratio of the hydroxy form compounds present in the internal olefin sulfonates (A) and (B) to the olefin form compounds present in the internal olefin sulfonates (A) and (B) is from 75/25 to 100/0.

By “internal olefin sulfonate”, we mean the reaction product obtained by sulfonation reaction of an internal olefin, followed by a neutralization step and then a hydrolysis step.

By “internal olefin”, we mean an olefin in which the double bond C═C is located internally in the chain (which means not located at the extremity of the said chain).

By “hydroxy form compound”, we mean the saturated compounds comprising a hydroxy radical and a sulfonate radical, present in the said reaction product.

By “olefin form compound”, we mean the unsaturated compounds comprising a C═C double bond and a sulfonate radical, present in the said reaction product.

Preferably, the sulfonate mixture comprises the internal olefin sulfonate (A) and the internal olefin sulfonate (B) in an amount from 50 to 100% by weight relative to the total weight of the sulfonate mixture.

Preferably, the sulfonate mixture of the invention comprises the internal olefin sulfonates (A) and (B) having a sulfonate group on the carbon atom located at C-2 position in a total amount of 28% by weight or less relative to the total weight of the sulfonate mixture. More preferably, it is from 10 to 28% by weight, and better from 15 to 25% by weight.

Preferably, the weight ratio (A/B) of the internal olefin sulfonate (A) to the internal olefin sulfonate (B) present in the sulfonate mixture is from 80/20 to 85/15.

Preferably, the weight ratio of the hydroxy form compounds present in the said internal olefin sulfonates (A) and (B) to the olefin form compounds present in the internal olefin sulfonates (A) and (B) is from 80/20 to 95/5.

The internal olefin sulfonate (A) having 16 carbon atoms can therefore contain one or more hydroxyl form compounds and optionally one or more olefin form compounds.

The internal olefin sulfonate (A) having 18 carbon atoms can therefore contain one or more hydroxyl form compounds and optionally one or more olefin form compounds.

The sulfonate mixture used in the context of the present invention can also be defined as comprising:

one or more compounds of formula (A1):

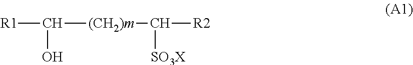

in which:

R1 and R2 represent, independently, a hydrogen atom or a branched or linear C₁-C₁₄ alkyl radical;

m is an integer from 0 to 14,

R1, R2 and m are such that the said compound(s) (A1) comprise 16 carbon atoms and

X represents a cation or a mixture of cations allowing achievement of electroneutrality of the said compound(s) (A1);

one or more compounds of formula (B1):

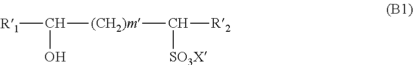

in which:

R′1 and R′2 represent, independently, a hydrogen atom or a branched or linear

C₁-C₁₆ alkyl radical;

m′ is an integer from 0 to 16,

R′1, R′2 and m′ being such that the said compound(s) (B1) comprise 18 carbon atoms and

X′ represents a cation or a mixture of cations allowing achievement of electroneutrality of the said compound(s) (B1); and

optionally one or more compounds of formula (A2) and/or (A3):

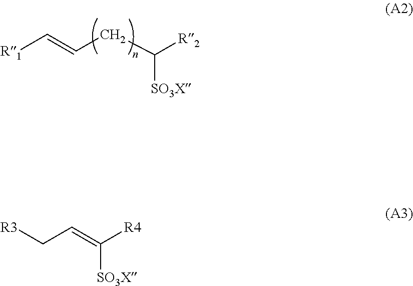

in which:

R″1, R″2, R3 and R4 represent, independently, a hydrogen atom or a branched or linear C₁-C₁₃ alkyl radical;

n is an integer from 0 to 13,

R″1, R″2, n, R3 and R4 being such that the said compound(s) (A2) and (A3) comprise 16 carbon atoms, and

X″ represents a cation or a mixture of cations allowing to achieve electroneutrality of the said compound(s) (A2) and/or (A3); and

optionally one or more compounds of formula (B2) and/or (B3):

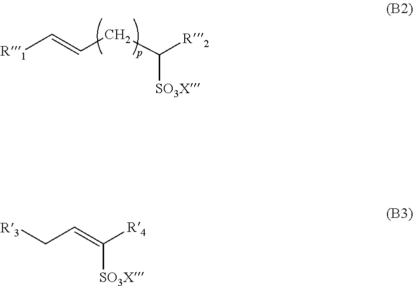

in which:

R′″1, R′″2, R′3 and R′4 represent, independently, a hydrogen atom or a branched or linear C₁-C₁₅ alkyl radical;

p is an integer from 0 to 15,

R′″1, R′″2, p, R′3 and R′4 being such that the said compound(s) (B2) and (B3) comprise 18 carbon atoms, and

X′″ represents a cation or a mixture of cations allowing achievement of electroneutrality of the said compound(s) (B2) and/or (B3);

wherein:

the weight ratio of all the compounds (A1), (A2) and (A3) to all the compounds (B1), (B2) and (B3) (which means (A1+A2+A3)/(B1+B2+B3)) is from 75/25 to 90/10; and

the weight ratio of all the compounds (A1) and (B1) to all the compounds (A2), (B2), (A3) and (B3) (which means (A1+B1)/(A2+A3+B2+B3)) is from 75/25 to 100/0.

In this definition, the compounds of formula (A1) and (B1) correspond to the hydroxy form compounds.

In this definition, the optional compounds of formula, (A2), (B2), (A3) and (B3) correspond to the olefin form compounds.

In the present application, in the calculation of the weight ratios, by “all the compounds (A1)”, we mean all the compounds corresponding to the formula (A1) which are present in the sulfonate mixture. It is the same for the other compounds (A2), (A3), (B1), (B2) and (B3).

Preferably, in the sulfonate mixture according to the invention, the weight ratio of all the compounds (A1), (A2) and (A3) to all the compounds (B1), (B2) and(B3) (which means (A1+A2+A3)/(B1+B2+B3)) is from 80/20 to 85/15.

Preferably, in the sulfonate mixture according to the invention, the weight ratio of all the compounds (A1) and (B1) to all the compounds (A2), (B2), (A3) and (B3) (which means (A1+B1)/(A2+A3+B2+B3)) is from 80/20 to 95/5, preferably from 85/15 to 90/10.

Preferably, the alkyl radicals (ie: R1, R2, R′1, R′2, R″1, R″2, R3, R4, R′″1, R′″2, R′3 and R′4) are linear.

Preferably, the cations X, X′, X″ and X′″ are organic or inorganic. They are preferably selected such that the compounds (A1), (A2), (A3), (B1), (B2) and (B3) are physiologically acceptable, in particular cosmetically acceptable.

In the sense of the invention, by “physiologically acceptable compound”, we mean a compound suitable for the topical administration of a composition containing it.

Preferably, the cations are selected from those corresponding to alkaline metals or alkalino-earth metals; in particular, selected from those corresponding to alkaline metals, and better corresponding to sodium (Na⁺).

Preferably, the sulfonate mixture according to the invention comprises at least compounds of formula (A1) in which m=0, the group (CH₂)ₘ being therefore replaced by a carbon-carbon simple bond (C—C).

Preferably, the sulfonate mixture according to the invention comprises at least compounds of formula (B1) in which m′=0, the group (CH₂)_{m′} being therefore replaced by a carbon-carbon simple bond (C—C).

Preferably, when the sulfonate mixture according to the invention comprises also compounds of formula (A2), at least a part of the said compounds is such that n=0, the group (CH₂)ₙ being therefore replaced by a carbon-carbon simple bond (C—C).

Preferably, when the sulfonate mixture according to the invention comprises also compounds of formula (B2), at least a part of the said compounds is such that p=0, the group (CH₂)ₚ being therefore replaced by a carbon-carbon simple bond (C—C).

Preferably, the sulfonate mixture according to the invention, comprises at least compounds of formula (A1) in which R2=CH₃ (the sulfonate group being therefore located on the carbon atom at C2 position) and/or the sulfonate mixture according to the invention, comprises at least compounds of formula (B1) in which R′2=CH₃ (the sulfonate group being therefore located on the carbon atom at C2 position).

Preferably, the sulfonate mixture according to the invention, comprises the compounds having a sulfonate group located on the carbon atom at C2 position (in particular (A1), (B1) and optionally (A2), (B2), (A3), (B3)) in a total amount of 28% by weight or less relative to the total weight of the sulfonate mixture. More preferably, it is from 10 to 28% by weight, and better from 15 to 25% by weight.

Preferably, the sulfonate mixture comprises:

the compound(s) (A1) in an amount of from 50 to 90% by weight, preferably from 55 to 85% by weight, more preferably from 60 to 80% by weight, relative to the total weight of the sulfonate mixture and/or

the compound(s) (B1) in an amount of from 5 to 25% by weight, preferably from 10 to 23% by weight, more preferably from 15 to 20% by weight, relative to the total weight of the sulfonate mixture and/or

the compound(s) (A2) and (A3) in a total amount of from 0 to 20% by weight, preferably from 1 to 20% by weight, more preferably from 5 to 15% by weight, relative to the total weight of the sulfonate mixture and/or

the compound(s) (B2) and (B3) in a total amount of from 0 to 7% by weight, preferably from 0.5 to 5% by weight, more preferably from 1 to 4% by weight, relative to the total weight of the sulfonate mixture.

The sulfonate mixture according to the invention may be obtained by sulfonation of an internal olefin, followed by a neutralization step then by a hydrolysis step.

A general method of preparation of the internal olefin sulfonate is notably described in the publication ‘Hydroxy alkane sulfonate, a new surfactant based on olefins’, by Stapersma et al., published in JAOCS, vol. 69, no 1 (January 1992).

Advantageously, a composition of the invention is comprised preferably from 0.5% to 20% by weight of the internal olefin sulfonates, more preferably from 1% to 15% by weight, more particularly from 1% to 10% by weight relative to the total weight of the composition.

Foam-Enhancing Agents or Foam Boosters

According to one embodiment of the invention, the compositions may further contain at least one foam-enhancing agent or foam boosters.

The foam-enhancing agents or foaming boosters which are suitable according to the invention can be preferably selected from:

the fatty alcohols;

the polyalkyleneglycols;

the polyglyceryl fatty acid esters;

the polyalkyleneglycol ethers of alkylglucose;

the celluloses;

the crosslinked polymers formed by the reaction of an C₁₀-C₁₈ alkylglucoside with 1,3-dichloro-2-propanol, and sulfonated with 3-chloro-2-hydroxypropyl sulfonate;

the fatty acid alkanolamides;

and their mixtures.

A) The fatty alcohols are preferably those comprising a linear saturated alkyl chain having from 10 to 18 carbon atoms as for instance lauryl alcohol, myristyl alcohol, cetyl alcohol, stearyl alcohol and their mixtures like mixtures of cetyl alcohol and stearyl alcohol (cetearyl alcohol);

B) The polyalkyleneglycols are preferably those of formula (II)

H—[O—R-]n-OH (II)

in which

R represents a linear alkyl chain containing from 1 to 4 carbon atoms, and

n is an integer ranging from 4 to 100000 and advantageously from 4 to 50000.

According to one particular embodiment of the invention, a polyalkylene glycol in accordance with the invention is a polyethyleneglycol.

According to one preferred embodiment, a polyethylene glycol in accordance with the invention may be chosen from PEG-8 such as the product sold under the trade name Polyethylene Glycol 400 DUB® by the company Clariant, PEG-45M such as the product sold under the trade name POLYOX WSR N 60 K® by the company Dow Chemical.

C) The polyglyceryl fatty acid esters are preferably monoesters of one fatty acid comprising a saturated alkyl chain having from 10 to 18 carbon atoms and a polyglycerol having from 2 to 30 moles of glycerol groups.

According to one preferred embodiment, a polyglycerolated ester in accordance with the invention may be chosen from POLYGLYCERYL-2 LAURATE, such as the product sold under the trade name SUNSOFT Q-12D-C® by the company TAIYO KAGAKU.

D) The polyalkyleneglycol ethers of alkylglucose and preferably polyC₁-C₄alkyleneglycol ethers of C₁-C₄alkylglucose especially Methyl Gluceth-10 as the product sold under the trade name GLUCAM E-10 HUMECTANT® by the company LUBRIZOL and Methyl Gluceth-20 as the product sold under the trade name GLUCAM E-20 HUMECTANT® by the company LUBRIZOL.

E) The celluloses are preferably polyalkyleneglycol ethers of alkylcellulose like hydroxypropyl methylcellulose as the product sold under the trade name BENECEL K100M HYDROXYPROPYLMETHYL CELLULOSE® by the company ASHLAND.

F) the crosslinked polymers formed by the reaction of an C₁₀-C₁₈ alkylglucoside with 1,3-dichloro-2-propanol, and sulfonated with 3-chloro-2-hydroxypropyl sulfonate are selected preferably from the polymers having their respective INCI name:
SODIUM HYDROXYPROPYLSULFONATE LAURYLGLUCOSIDE CROSSPOLYMER sold under the trade name POLYSUGA NATE 160P® by the company COLONIAL CHEMICAL INC.
SODIUM HYDROXYPROPYLSULFONATE COCOGLUCOSIDE CROSSPOLYMER sold under the trade name POLY SUGANATE 124P® by the company COLONIAL CHEMICAL INC.
SODIUM HYDROXYPROPYLSULFONATE DECYLGLUCOSIDE CROSSPOLYMER sold under the trade name POLY SUGANATE 100P® by the company COLONIAL CHEMICAL INC.

G) the fatty acid alkanolamides are preferably selected from C₁₂-C₁₈ fatty acid alkanolamides like Cocamide MEA as the product sold under the trade name COMPERLAN CMEA® by the company BASF.

The most preferred foam boosters will be selected from the polylethyleneglycols and polyglyceryl fatty acid esters and more particularly selected from PEG-45M and POLYGLYCERYL-2 LAURATE.

Advantageously, a composition of the invention comprises preferably from 0.05% to 10% by weight of the foam-enhancing agent or foam booster, more preferably from 0.1% to 5% and much more preferably from 0.2% to 3% by weight by weight relative to the total weight of the composition.

According to one embodiment of the invention, the compositions will be sulfate surfactant-free.

For the purposes of the present invention, the term “sulfate surfactant-free” means that the composition contains less than 1.0% by weight relative to the total weight of the composition of surfactant of the type sulphate (ie alkylsulfates, alkylether sulfates) and preferably less than 0.5% by weight and more preferably less than 0.1% by weight.

According to one embodiment of the invention, the compositions may further contain other surfactants which are selected from anionic surfactants, non-ionic surfactants, amphoteric or zwitterionic surfactants, cationic surfactants and their mixtures.

Soaps

According to one embodiment of the invention, the compositions may further contain at least one fatty acid salt (soap).

The fatty acid salt or soap according to the invention is obtained from a fatty acid and a base, the fatty acid comprising preferably a saturated or unsaturated, linear or branched alkyl chain having from 12 to 22 carbon atoms and preferably 12 to 20 carbon atoms.

The bases (also known as saponifiers) partially or completely neutralize the fatty acids. The bases capable of being used to obtain the salts may be, for example, inorganic bases such as alkali metal hydroxides (sodium hydroxide and potassium hydroxide), alkaline-earth metal hydroxides (magnesium hydroxide) or ammonium hydroxide, or else organic bases such as triethanolamine, N-methylglucamine, lysine and arginine.

The degree of neutralization of the fatty acid is defined as being the weight ratio between the fatty acids in the form of salts and the total fatty acids (free acids plus fatty acid salts). In the composition according to the invention, this degree of neutralization preferably ranges from 80 to 110%, and better still from 85 to 100%.

According to one particular embodiment of the invention, the base is potassium hydroxide.

The soap is generally introduced into the composition in the form of the base on the one hand and of the fatty acid on the other hand, the formation of the salt taking place in situ.

The fatty acid may be chosen, in particular, from C₁₀ to C₂₄ and especially C₁₂-C₁₈ fatty acids and in particular lauric acid, myristic acid, stearic acid, palmitic acid and mixtures thereof.

As soaps, mention may be made, for example, of the potassium salts and the sodium salts of C₁₀-C₂₄, especially C₁₂-C₂₀, more especially C₁₂-C₁₈ fatty acids. The soap may be more especially the sodium salts of C₁₂-C₁₈ fatty acids, more especially the potassium salts of lauric acid, stearic acid, palmitic acid and their mixtures.

The total amount of fatty acid salts in the composition of the invention varies preferably from 0.5 to 40% by weight and better still from 1 to 40% by weight and more preferably from 2 to 30% by weight relative to the total weight of the composition.

Amphoteric or Zwitterionic Surfactants

According to one embodiment of the invention, the compositions may further contain at least one amphoteric or zwitterionic surfactant.

The amphoteric or zwitterionic surfactants can be chosen, for example, from betaines, N-alkylamidobetaines and derivatives thereof, sultaines, alkyl polyaminocarboxylates, alkylamphoacetates, and mixtures thereof.

Mention may in particular be made, as betaines, of alkyl betaines, such as, for example, Coco betaine, such as the product sold under the name Dehyton AB-30® by the company Cognis, Lauryl betaine, such as the product sold under the name Genagen KB® by the company Clariant, oxyethylenated (10 EO) lauryl betaine, such as the product sold under the name Lauryl Ether (10 EO) Betaine® by the company Shin Nihon Rica, or oxyethylenated (10 EO) stearyl betaine, such as the product sold under the name Stearyl Ether (10 EO) Betaine® by the company Shin Nihon Rica.

Mention may be made, among N-alkylamidobetaines and derivatives thereof, for example, of Cocamidopropyl betaine, sold under the name LEBON 2000 HG® by the company Sanyo or sold under the name EMPIGEN BB® by the company Albright & Wilson, or lauramidopropyl betaine, sold under the name Rewoteric AMB12P® by the company Witco.

Mention may be made, as sultaines, of hydroxysultaines, cocamidopropyl hydroxysultaine, such as the product sold under the name REWOTERIC AM CAS® by the company Goldschmidt-Degussa or the product sold under the name CROSULTAINE C-50® by the company Croda.

Mention may be made, as alkyl polyaminocarboxylates (APACs), of sodium cocoylpolyaminocarboxylate, sold under the names AMPHOLAK 7 CX/C® and Ampholak 7 CX® by the company Akzo Nobel, sodium stearylpolyamidocarboxylate, sold under the name AMPHOLAK 7 TX/C® by the company Akzo Nobel, or sodium carboxymethyloleylpolypropylamine, sold under the name Ampholak XO7/C® by the company Akzo Nobel.

Mention may be made, as alkylamphoacetates, for example, of N-disodium N-cocoyl-N-carboxymethoxyethyl-N-(carboxymethyl)ethylenediamine (CTFA name: disodium cocoamphodiacetate), such as the product sold under the name Miranol C2M Concentré NP® by the company Rhodia, N-sodium N-cocoyl-N-hydroxyethyl-N-(carboxymethyl)ethylenediamine (CTFA name: sodium cocamphoacetate) or sodium cocoamphohydroxypropylsulfonate, sold under the name Miranol CSE by the company Rhodia.

Preferably, the amphoteric or zwitterionic surfactant is chosen from betaines, and especially alkylbetaines and more particularly Coco betaine, such as the product sold under the name DEHYTON AB-30® by the company Cognis.

Advantageously, a composition of the invention comprises, preferably from 0.1% to 20% by weight of the amphoteric or zwitterionic surfactant, more preferably from 0.5% to 15% by weight, more particularly from 1% to 10% by weight relative to the total weight of the composition.

According to one particular embodiment of the invention, the composition contains in an aqueous medium:

a) at least one internal olefin sulfonates composition as above defined; and

b) at least one enhancing-foam agent or foam booster as above defined; and

c) at least one fatty acid salt as above defined; and

d) at least one amphoteric or zwitterionic surfactant as above defined.

According to one particular embodiment of the invention, the composition contains in an aqueous medium:

a) at least one internal olefin sulfonates composition as above defined in an amount of from 0.5% to 20% by weight relative to the total weight and

b) at least one enhancing-foam agent or foam booster as above defined in an amount of from 0.05% to 10% by weight relative to the total weight of the composition, and

c) at least one fatty acid salt as above defined in an amount from 0.5% to 40% by weight relative to the total weight of the composition, and

d) at least one amphoteric or zwitterionic surfactant as above defined in an amount of from 0.1% to 20% by weight relative to the total weight of the composition.

According to one particular embodiment of the invention, the composition contains in an aqueous medium:

a) at least one internal olefin sulfonates composition as above defined in an amount of from 1% to 15% by weight relative to the total weight; and

b) at least one enhancing-foam agent or foam booster as above defined in an amount of from 0.1% to 5% by weight relative to the total weight of the composition; and

c) at least one fatty acid salt as above defined in an amount from 1% to 40% by weight relative to the total weight of the composition; and

d) at least one amphoteric or zwitterionic surfactant as above defined in an amount of from 0.15% to 15% by weight relative to the total weight of the composition.

According to one particular embodiment of the invention, the composition contains in an aqueous medium:

a) at least one internal olefin sulfonates composition as above defined in an amount of from 1% to 10% by weight relative to the total weight; and

b) at least one enhancing-foam agent or foam booster as above defined in an amount of from 0.2% to 3% by weight relative to the total weight of the composition; and

c) at least one fatty acid salt as above defined in an amount from 2% to 30% by weight relative to the total weight of the composition; and

d) at least one amphoteric or zwitterionic surfactant as above defined in an amount of from 1% to 10% by weight relative to the total weight of the composition.

According to one preferred embodiment of the invention, the said compositions are sulfate surfactant-free.

Polymeric Suspension Agent

According to one embodiment of the invention, the compositions may further contain at least one polymeric suspending agent which is different from the foam enhancing agent or foam booster.

For the purposes of the present invention, the term “polymeric suspension agent” means any polymeric compound which gels or thickens the composition and also allow to avoid sedimentation of the eventual particles which may be present in the composition according to the present invention.

For the purposes of the present invention, the term “polymeric compound” means any large molecule, or macromolecule, composed of repeated subunits, known as monomers (at least two monomers) which are linked with each other by covalent bonds by chemical reaction.

The polymeric suspension agents may be of natural origin. They may then be chosen from water-soluble polysaccharides polymers.

The term “polysaccharide” means any polymer consisting of several saccharides (or monosaccharides) having the general formula:

—[Cₓ(H₂O)]ₙ— (in which y is generally x−1)

and linked together via O-oside bonds.

The term “water-soluble polysaccharide” means any polysaccharide which, when introduced into water or into a mixture of water and linear or branched C₂-C₅ monoalcohols, for instance ethanol, isopropanol or n-propanol, without pH modification at 25° C., to a mass concentration equal to 1%, allows the production of a macroscopically homogeneous and transparent solution, i.e. a solution with a minimum light transmission value, at a wavelength equal to 500 nm, through a sample 1 cm thick, of at least 80% and preferably at least 90%.

The water-soluble polysaccharides that may be used in the present invention are especially chosen from starches, carrageenan, xanthan gum, guar gum, and celluloses such as hydroxyethylcellulose and hydroxypropylcellulose, and mixtures thereof.

The polymeric suspension agents according to the invention may be of synthetic origin.

They may be chosen from

the associative or non-associative crosslinked homopolymers and crosslinked copolymers of acrylic acid and/or methacrylic acid and/or their esters;

the non-ionic associative polyether-urethanes.

For the purposes of the present invention, the term “associative polymers” means hydrophilic polymers that are capable, in an aqueous medium, of reversibly associating with each other or with other molecules. Their chemical structure more particularly comprises at least one hydrophilic region and at least one hydrophobic region.

The term “hydrophobic group” is understood to mean a radical or polymer comprising a saturated or unsaturated and linear or branched hydrocarbon-based chain. When the hydrophobic group denotes a hydrocarbon-based radical, it comprises at least 10 carbon atoms, preferably from 10 to 30 carbon atoms, in particular from 12 to 30 carbon atoms and more preferentially from 18 to 30 carbon atoms. Preferentially, the hydrocarbon-based group is derived from a monofunctional compound.

By way of example, the hydrophobic group may be derived from a fatty alcohol, such as stearyl alcohol, dodecyl alcohol or decyl alcohol, or else from a polyoxyalkylenated fatty alcohol, such as Steareth-100. It may also denote a hydrocarbon-based polymer, for instance polybutadiene.

For the purposes of the present invention, the term “non associative polymers” means that the polymer does not have the behaviour of an associative polymer as above explained. It is generally a hydrophilic polymer of acrylic acid and/or methacrylic acid and of an ester thereof comprising less than 6 carbon atoms: ie a C₁-C₄ alkylacrylate for example, chosen from methyl acrylate, ethyl acrylate and butyl acrylate.

The copolymers disclosed herein are partially or totally crosslinked with at least one standard crosslinking agent. The at least one crosslinking agent can be chosen, for example, from polyunsaturated compounds, such as polyethylenically unsaturated compounds. For example, these compounds can be chosen from polyalkenyl ethers of sucrose, polyalkenyl ethers of polyols, diallyl phthalates, divinylbenzene, allyl (meth)acrylate, ethylene glycol di(meth)acrylate, methylenebisacrylamide trimethylolpropane tri(meth)acrylate, diallyl itaconate, diallyl fumarate, diallyl maleate, zinc (meth)acrylate, castor oil derivatives and polyol derivatives manufactured from unsaturated carboxylic acids.

As examples of such polymers, may be mentioned:

(1) The homopolymers of acrylic acid crosslinked with an allyl ether of pentaerythritol, an allyl ether of sucrose, or an allyl ether of propylene like the Carbomer sold under the commercial name CARBOPOL® by the company LUBRIZOL.

(2) The non-associative crosslinked copolymers of acrylic acid and/or methacrylic acid and esters thereof.

As an example of such non-associative crosslinked copolymers of acrylic acid and/or methacrylic acid and esters thereof, can be mentioned:

i) The crosslinked copolymers of acrylic acid and/or methacrylic acid and of an ester thereof comprising less than 6 carbon atoms and more particularly in the form of an aqueous 30% dispersion as the copolymer sold under the commercial name ACULYN 33® by the company THE DOW CHEMICAL COMPANY and having the INCI name: ACRYLATES COPOLYMER.

ii) The crosslinked copolymers comprising at least one methacrylic acid unit and at least one C₁-C₄ alkyl acrylate unit. These copolymers are described, for example, in Patent Application no WO 01/76552.

As used herein, the crosslinked copolymer comprising at least one methacrylic acid unit and at least one C₁-C₄alkyl acrylate unit means a crosslinked copolymer comprising at least one methacrylic acid unit and at least one alkyl acrylate unit, wherein the alkyl acrylate unit is chosen from C₁-C₄ alkyl acrylates.

In the crosslinked copolymers disclosed herein, the methacrylic acid unit can be present, for example, in an amount ranging from 20% to 80% by weight, such as from 25% to 70% by weight, and further such as from 35% to 60% by weight, relative to the total weight of the copolymer.

In the crosslinked copolymer disclosed herein, the alkyl acrylate unit can be present, for example, in an amount ranging from 15% to 80% by weight, such as from 25% to 75% by weight and further such as from 40% to 65% by weight, relative to the total weight of the copolymer. The alkyl acrylate unit can be chosen, for example, from methyl acrylate, ethyl acrylate and butyl acrylate. In one embodiment, the alkyl acrylate unit is ethyl acrylate.

According to one embodiment, the at least one crosslinked copolymer disclosed herein may be in the form of a dispersion in water. The number-average size of the copolymer particles in the dispersion generally ranges from 10 to 500 nm, for example, from 20 to 200 nm and further, for example, from 50 to 150 nm.

Use can be made, for example, of the crosslinked copolymers comprising at least one methacrylic acid unit and at least one ethyl acrylate unit in the form of an aqueous 30% dispersion manufactured and sold under the name CARBOPOL AQUA SF-1® by the company NOVEON.

Amongst the polymeric suspension agents chosen from crosslinked homopolymers and crosslinked copolymers of acrylic acid and/or methacrylic acid and/or their esters can be also mentioned:

(3) The associative anionic crosslinked polymers of acrylic acid and/or methacrylic acid and/or their esters and more particularly those consisting of 95 to 60% by weight of acrylic acid (hydrophilic unit), 4 to 40% by weight of C₁₀-C₃₀ alkyl acrylate (hydrophobic unit), and 0 to 6% by weight of crosslinking polymerizable monomer, or alternatively those consisting of 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer such as those described above.

Among said above polymers, can be cited the products sold by the company GOODRICH under the trade names PEMULEN TR1®, PEMULEN TR2®, CARBOPOL 1382®, and still more preferably PEMULEN TR1®, and the product sold by the company S.E.P.P.I.C. under the name COATEX SX®, and having the INCI name: ACRYLATES/C₁₀-C₃₀ ALKYL ACRYLATE CROSSPOLYMER.

We can also mention the copolymers comprising among their monomers a carboxylic acid with α,β-monoethylenic unsaturation and an ester of a carboxylic acid with α,β-monoethylenic unsaturation and an oxyalkylenated fatty alcohol. Preferably, these compounds also comprise, as monomer, an ester of a carboxylic acid with α,β-monoethylenic unsaturation and a C₁-C₄ alcohol. By way of example of this type of compound, there may be mentioned ACULYN 22® sold by the company THE DOW CHEMICAL COMPANY which is an oxyalkylenated stearyl methacrylate/ethyl acrylate/methacrylic acid terpolymer with the INCI Name ACRYLATES/STEARETH-20 METHACRYLATE COPOLYMER.

We can also mention the crosslinked copolymers of acrylic acid/vinyl isodecanoate as product sold under the commercial name STABYLEN 30® (INCI name: ACRYLATES/VINYL ISODECANOATE CROSSPOLYMER) sold by the company SIGMA 3V.

We can also mention the crosslinked copolymers of vinyl neodecanoate and one or more monomers of acrylic acid, methacrylic acid or one of their simple esters crosslinked with an allyl ether of trimethylolpropane or pentaerythritol as the product sold under the commercial name ACULYN 38® (INCI name: ACRYLATES/VINYL NEODECANOATE CROSSPOLYMER) sold by THE DOW CHEMICAL COMPANY.

Another family of polymeric suspension agent which is suitable for the composition of the invention is composed of the non-ionic associative polyether-urethanes.

Non-ionic associative polyether-urethanes are nonionic block copolymers comprising in the chain both hydrophilic blocks usually of polyoxyethylene nature (polyurethanes may also be referred to as polyurethane polyethers), and hydrophobic blocks that may be aliphatic sequences alone and/or cycloaliphatic and/or aromatic sequences.

In particular, these polymers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be provided. In addition, the polymer may comprise a hydrocarbon-based chain at one end or at both ends of a hydrophilic block.

Associative polyurethanes may be block polymers, in triblock or multiblock form. The hydrophobic blocks may thus be at each end of the chain (for example: triblock copolymer containing a hydrophilic central block) or distributed both at the ends and in the chain (for example: multiblock copolymer). These polymers may also be graft polymers or star polymers. Preferably, the associative polyurethanes are triblock copolymers in which the hydrophilic block is a polyoxyethylene chain comprising from 50 to 1000 oxyethylene groups. In general, associative polyurethanes comprise a urethane bond between the hydrophilic blocks, whence arises the name.

As examples of nonionic fatty-chain polyurethane polyethers that may be used in the invention, it is also possible to use Rheolate® FX 1100 (Steareth-100/PEG 136/HDI (hexamethyl diisocyanate) copolymer), Rheolate® 205® containing a urea function, sold by the company Elementis, or Rheolate® 208, 204 or 212.

Mention may also be made of the product Elfacos T210® containing a C12-C14 alkyl chain, and the product Elfacos T212® containing a C16-18 alkyl chain (PPG-14 Palmeth-60 Hexyl Dicarbamate) from Akzo.

The associative polyurethanes that may be used according to the invention are in particular those described in the article by G. Fonnum, J. Bakke and Fk. Hansen—Colloid Polym. Sci., 271, 380-389 (1993).

Even more particularly, according to the invention, use may also be made of a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyether-urethanes are sold especially by the company THE DOW CHEMICAL COMPANY under the names Aculyn 46® and Aculyn 44®. Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Use will be made more particularly of an associative nonionic polyurethane polyether such as the product sold especially by the company Elementis under the name Rheolate FX 1100®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 30,000 (INCI name: PEG-136/Steareth-1001/SMDI Copolymer).

According to a preferred embodiment of the invention, the polymeric suspension agent will be chosen from the non-associative crosslinked copolymers of acrylic acid and/or methacrylic acid and esters thereof and more preferably:

the crosslinked copolymers comprising at least one methacrylic acid unit and at least one ethyl acrylate unit and more particularity in the form of an aqueous 30% dispersion manufactured and sold under the name CARBOPOL AQUA SF-1® by the company NOVEON;

the crosslinked copolymers of acrylic acid and/or methacrylic acid and of an ester thereof comprising less than 6 carbon atoms and more particularity in the form of an aqueous 28% dispersion as the copolymer sold under the commercial name ACULYN 33® by the company THE DOW CHEMICAL COMPANY and having the INCI name: ACRYLATES COPOLYMER and their mixtures.

The polymeric suspension agent may be included in the composition according to the present invention, preferably from 0.1% to 10% by weight relative to the total weight of the composition, more preferably from 1 to 8% by weight, and more particularly from 2 to 5% by weight relative to the total weight of the composition.

Aqueous Phase

The composition according to the invention comprises an aqueous medium or aqueous phase, i.e. a medium comprising an amount of water of at least 50% by weight, preferably ranging from 50% to 95% by weight and better still from 60% to 90% by weight relative to the total weight of the composition.

The aqueous phase of the compositions according to the invention may contain, besides water, one or more solvents chosen from monoalcohols comprising from 1 to 6 carbon atoms, and polyols, and mixtures thereof. A monoalcohol that may especially be mentioned is ethanol. Examples of polyols that may especially be mentioned include glycerol; glycols such as butylene glycol, isoprene glycol or propylene glycol, polyethylene glycols such as PEG-8; sorbitol; dialkyleneglycols as dipropyleneglycol; sugars such as glucose, fructose, maltose, lactose and sucrose; and mixtures thereof.

When they are present, the amount of monoalcohols and of polyols in the composition of the invention may range, for example, from 0.01% to 30% by weight, preferably from 2% to 25% by weight and better still from 4% to 20% by weight relative to the total weight of the composition.

The pH of the composition according to the invention is preferably from 7.0 to 10 and more preferably from 8 to 9.

Additives

The composition according to the invention may contain various additives, chosen from those conventionally used in skincare or makeup-removing products, insofar as these additives and the amounts thereof do not harm the desired qualities for the composition according to the invention.

The cleansing composition in accordance with the present invention may thus comprise the following additives: co-surfactants; oil; preserving agents; sequestrants (EDTA and salts thereof); alkalinizing or acidifying agents antioxidants; fragrances; dyestuffs; encapsulated or non-encapsulated pigments or soluble dyes; anionic, nonionic, cationic or amphoteric, polymers, exfoliating agents, peeling agents as citric acid, glycolic acid, hyaluronic acid; fillers, propellants.

The amounts of these various adjuvants are those conventionally used in the field under consideration, for example from 0.01% to 20% of active material of the total weight of the composition. These adjuvants and the amounts thereof should be such that they do not modify the property desired for the composition of the invention.

The composition may also comprise a polymeric quaternary ammonium salts. These compounds are conditioning agents, i.e. they produce a comfortable sensation of softness on the skin (moisturization maintenance).

The polymeric quaternary ammonium salts are cationic or amphoteric polymers containing at least one quaternized nitrogen atom. Polymeric quaternary ammonium salts that may especially be mentioned include the Polyquaternium products (CTFA name), which afford softness and creaminess to the foaming cream. These polymers may preferably be chosen from the following polymers:

Polyquaternium 5, such as the product Merquat 5® sold by the company Nalco;

Polyquaternium 6, such as the product Salcare SC 30® sold by the company Ciba, and the product Merquat 100® sold by the company Nalco;

Polyquaternium 7, such as the products Merquat S®, Merquat 2200 and Merquat 550 sold by the company Nalco, and the product Salcare SC 10® sold by the company Ciba;

Polyquaternium 10, such as the product Polymer JR400® sold by the company Amerchol;

Polyquaternium 11, such as the products Gafquat 755®, Gafquat 755N® and Gafquat 734® sold by the company ISP;

Polyquaternium 15, such as the product Rohagit KF 720 F® sold by the company Röhm;

Polyquaternium 16, such as the products Luviquat FC905®, Luviquat FC370®, Luviquat HM552® and Luviquat FC550® sold by the company BASF;

Polyquaternium 22, such as the product Merquat 280® sold by the company Nalco;

Polyquaternium 28, such as the product Styleze CC10® sold by the company ISP;

Polyquaternium 39, such as the products Merquat Plus 3330® and Merquat 3330PR® sold by the company Nalco;

Polyquaternium 44, such as the product Luviquat Care® sold by the company BASF;

Polyquaternium 46, such as the product Luviquat Hold® sold by the company BASF;

Polyquaternium 47, such as the product Merquat 2001® sold by the company Nalco.

Preferably, the quaternary ammonium salts are chosen from Polyquaternium-7, Polyquaternium-10, Polyquaternium-39 and Polyquaternium-47, and mixtures thereof.

The polymeric quaternary ammonium salts may be in an (active material) amount ranging, for example, from 0.01% to 5% by weight and better still from 0.05% to 1% by weight relative to the total weight of the composition.

As an example of a particular conditioning agent, mention may be made of

Polyquaternium-39, sold especially by the company Nalco under the names Merquat Plus 3330® and Merquat 3330PR®.

As an example of a particular conditioning agent, mention may be made of polyquaternium 10, such as the product Polymer JR400® sold by the company Amerchol.

Galenic Forms

The compositions according to the present invention may be under the form of any galenic form generally used in the products for removing makeup and/or for cleansing the skin, the hair and/or mucous.

They may be conditioned in various types of devices as tubes, bottles. They may be conditioned under a pressurized form as aerosols or non-aerosol pumps.

### EXAMPLES

### Example 1 of Preparation of a Sulfonate Mixture

### Example A

### Synthesis of C₁₆ Internal Olefins

7000 g (28.9 moles) of 1-hexadecanol (trade name: KALCOL 6098® of the company Kao Corporation), and, as a solid acid catalyst, 700 g (10% by weight relative to the weight of the raw material alcohol) of alumina (STREM Chemicals, Inc.) were placed into a flask with a stirrer, and reactions were allowed to proceed for three hours at 280° C. while stirring and passing nitrogen (7000 mL/minute) through the system. The alcohol conversion ratio was 100% and the purity of C₁₆ internal olefin was 99.6% after the completion of the reaction. The resulting crude internal olefin was transferred to a distillation flask and distilled at from 136 to 160° C./4.0 mmHg, whereby 100% of pure internal olefin having 16 carbon atoms was obtained. The double bond distribution in the resulting internal olefin was less than 1% by weight at C-1position, 27% by weight at C-2 position, 23% weight at C-3 position, 18% weight at C-4position, 16% weight at C-5 position, 8% weight at C-6 position, and 7% weight in total at C-7 and C-8 positions.

### Example B

### Synthesis of C₁₈ Internal Olefins

7000 g (25.9 moles) of 1-octadecanol (trade name: KALCOL 8098® of Kao Corporation), and, as a solid acid catalyst, 700 g (10% by mass relative to the raw material alcohol) of alumina (STREM Chemicals, Inc.) were placed into a flask with a stirrer, and reactions were allowed to proceed for 15 hours at 280° C. while stirring and passing nitrogen (7000 mL/minute) through the system. The alcohol conversion ratio was 100% and the purity of C₁₈ internal olefin was 98.2% after the completion of the reaction. The resulting crude internal olefin was transferred to a distillation flask and distilled at from 148 to 158° C./0.5 mmHg, whereby 100% of pure internal olefin was obtained. The double bond distribution in the resulting internal olefin was less than 1% weight at C-1 position, 19% weight at C-2 position, 18% weight at C-3 position, 17% weight at C-4 position, 15% weight at C-5 position, 12% by mass at C-6 position, 9% weight at C-7 position, and 10% weight in total at C-8 and C-9 positions.

### Example C

### Production of the Mixture C₁₆ Internal Olefins and C₁₈ Internal Olefins

11.9 kg of the C₁₆ internal olefin (Example A) and 3.1 kg of the C₁₈ internal olefin (Example B) were mixed to produce 15.0 kg of C₁₆/C₁₈ internal olefins (mass ratio 79.4/20.6). The double bond distribution of the resulting internal olefin was 1% weight at a C-1 position, 25% weight at a C-2 position, 22% weight at a C-3 position, 18% weight at a C-4 position, 16% weight at a C-5 position, 9% weight at a C-6 position, 4% weight at a C-7 position, 4% weight at a C-8 position, and 1% weight at a C-9 position.

Synthesis of the Sulfonate Mixture

Using a thin film sulfonation reactor having an outer jacket, the sulfonation reaction of the internal olefin having 16 and 18 carbon atoms (the content of an internal olefin in which a double bond was present at a C-2 position is 25% weight) obtained in Example C was carried out by passing through sulfur trioxide gas, while passing cooling water of 10° C. through the outer jacket of the reactor.

The molar ratio of SO₃/internal olefin for the sulfonation reaction was set at 1.10. The resulting sulfonation product was added to an alkaline aqueous solution prepared with 1.11 times the molar amount of sodium hydroxide relative to the theoretical acid value, followed by neutralization at below 30° C. for less than 0.5 hour while stirring. The resulting neutralized product was hydrolyzed by heating at 180° C. for one hour in an autoclave. And then, the hydrolyzed product was decolorized by hydrogen peroxide, whereby a sodium C₁₆/C₁₈ internal olefin sulfonate mixture was obtained.

The weight ratio of hydroxy form compounds (sodium hydroxyalkane sulfonate)/olefin form compounds (sodium olefin sulfonate) in the obtained sodium internal olefin sulfonate mixture was 87/13. Also, the content of the raw material internal olefin contained in the obtained sodium internal olefin sulfonate mixture was 1.3% by weight, while the content of inorganic compounds therein was 4.7% by weight. Also, the content of compounds having a sulfonate group located at a C-2 position, present in the internal olefin sulfonate mixture was 16% by weight.

### Examples 2 to 7

The following compositions were prepared. The amounts of the ingredients in an active material content were expressed in percentages by weight relative to the total weight of the composition.

| | Example 2 | Example 3 | Example 4 | Example 5 | Example 6 | Example 7 |
|---|---|---|---|---|---|---|
| Ingredients | (invention) | (Invention) | (Invention) | (Invention) | (Invention) | (Invention) |
| Internal olefins | 8.3 | 8.3 | 8.3 | 8.3 | 8.3 | 8.3 |
| sulfonates composition | | | | | | |
| of example 1 | | | | | | |
| Stearic acid | 14.3 | — | — | — | — | — |
| Palmitic acid | 0.9 | 0.4 | 0.4 | 0.4 | 0.4 | — |
| Lauric acid | 4.0 | 2.4 | 2.4 | 2.4 | 2.5 | — |
| Cocobetaine | — | 3.4 | 3.4 | 3.4 | 3.4 | — |
| Myristyl alcohol | 0.3 | 0.3 | 0.3 | 0.3 | — | 0.3 |
| Glycerin | 15.0 | 13.0 | 13.0 | 13.0 | 13.0 | 13.0 |
| Potassium Hydroxide | 7.9 | 4.5 | 4.5 | 4.5 | 4.5 | — |
| Acrylates Copolymer | — | 1.0 | 1.0 | — | 1.0 | 1.0 |
| (ACULYN 33 ®) | | | | | | |
| PEG-45M | — | 0.1 | — | — | — | — |
| (POLYOX WSR N 60 K ®) | | | | | | |
| Cocamide MEA | — | 1.0 | — | — | — | — |
| Polyglyceryl-2 Laurate | 0.5 | 0.5 | 0.5 | 0.5 | 0.5 | 0.5 |
| (SUNSOFT Q-12D-C ®) | | | | | | |
| Disodium EDTA | 0.3 | 0.2 | 0.2 | 0.2 | 0.2 | 0.2 |
| Polyquaternium-10 | 0.6 | | | | | |
| (POLYMER JR400 ®) | | | | | | |
| Polyquaternium-39 | | 2.0 | 1.0 | 1.0 | 1.0 | — |
| (MERQUAT PLUS | | | | | | |
| 3330 ®) | | | | | | |
| Sodium | — | — | 0.2 | — | — | — |
| Hydroxypropylsulfonate | | | | | | |
| Laurylglucoside | | | | | | |
| Crosspolymer | | | | | | |
| (POLYSUGA NATE | | | | | | |
| 160P ®) | | | | | | |
| Hydroxypropyl methyl | — | — | — | 0.1 | — | — |
| cellulose | | | | | | |
| (BENECEL K100M | | | | | | |
| HYDROXYPROPYLMETHYL | | | | | | |
| CELLULOSE ®) | | | | | | |
| Methyl Gluceth-10 | — | — | — | — | 0.2 | — |
| (GLUCAM E-10 | | | | | | |
| HUMECTANT ®) | | | | | | |
| Water | qs | qs | qs | qs | qs | qs |
| pH | 8.9 | 8.8 | 8.9 | 8.7 | 8.9 | 8.9 |
| Foam quality | ++++ | +++ | +++ | +++ | +++ | ++ |
| Skin feel after rinsing off | fresh feel, | moisture | good film | moisture | not-tight, | moisture |
| | not-tight | feel | feel | feel | moisture | feel |
| | | | | | feel | |

Preparation of the Compositions

Example 2 was prepared with an agitator under vacuum. Examples 3, 4, 5, and 6 were prepared with a propeller.

(1) Fatty acids and glycerine were mixed at 80° C.

(2) KOH was added little by little into (1) at 80° C. while agitating to neutralize fatty acid.

(3) The mixture of internal olefin sulfonates, water, acrylate polymer (if needed) was prepared at room temperature, and added to (1)+(2), and agitated at 70-75° C.

(4) Foam boosters and water were mixed at 70-75° C., then added to (1)+(2)+(3) while agitating.

(5) The obtained composition was cooled down to room temperature.

The evaluation of both foam quality and skin feel after rinsing off was performed by adding 1 g of product to the hand and systematically creating a foam by introducing tap water. The prototype was mixed for 2 minutes by periodically adding water. After rinsing, the skin feel after rinsing off was also evaluated with a particular focus on whether the performance was squeaky, dry/tight or moisturized feeling. Biore skincare facial wash moisture was used as the positive control.

++++=very good foam quality regarding bubble size, volume and density.

+++=good foam quality regarding bubble size, volume and density.

++=moderate foam quality regarding bubble size, volume and density

### Comparative Tests Between Soap-Containing Formulations

### Examples 2 and 7 (Invention) and 8 (Outside the Invention)

A simple foaming test was conducted to examine the sulphate-free example 2 according to the invention containing the internal olefins sulfonates composition of example 1 compared to the example 8 (outside the invention) which contains a sulfate surfactant.

The foaming process was conducted as follows: 1 g of foaming cleanser was well dissolved in 20 g of tap water. Foam was generated systematically using a cappuccino (HARIO) machine for 20 seconds by placing the tip in the center of the beaker. The foam density (g/L) was measured with a specific container, and weighted just after the foam was generated. The method was repeated three times for each cleanser, and the data is tabulated in the Table below. A high foam density correlates to a smaller bubble size, which can possibly be linked to better foaming performance.

| | | | Example 8 |
|---|---|---|---|
| | Example 2 | Example 7 | (outside |
| Ingredients | (invention) | (invention) | invention) |
| Internal olefins sulfonates composition | 8.3 | 8.3 | — |
| of example 1 | | | |
| Sodium Laureth Sulfate (2OE) | — | — | 8.3 |
| Stearic acid | 14.3 | — | 14.3 |
| Palmitic acid | 0.9 | — | 0.9 |
| Lauric acid | 4.0 | — | 4.0 |
| Myristyl alcohol | 0.3 | 0.3 | 0.3 |
| Glycerin | 15.0 | 13.0 | 15.0 |
| Potassium Hydroxide | 7.9 | — | 7.9 |
| Polyglyceryl-2 Laurate | 0.5 | 0.5 | 0.5 |
| (SUNSOFT Q-12D-C ®) | | | |
| Disodium EDTA | 0.3 | 0.3 | 0.3 |
| Polyquaternium-10 | 0.6 | — | 0.6 |
| (POLYMER JR400 ®) | | | |
| Polyquaternium-39 | | | |
| (MERQUAT PLUS 3330 ®) | | | |
| Potassium Hydroxide | 0.81 | — | 0.81 |
| Water | qs | qs | qs |
| pH | 8.9 | 8.9 | 8.9 |
| Foam density (g/L) | 149.93 ± 1.12 | 136.52 ± 0.69 | 136.52 ± 0.69 |
| Foam quality (made by hand) | ++++ | +++ | +++ |
| Skin feel after rinsing off | fresh feel, | moisture feel | fresh feel |
| | not-tight | | |

We observed that composition 2 according to the invention containing the sulfonate composition and the foaming booster have a better foam density compared to the composition 8 (outside the invention) containing a sulfate surfactant instead of the sulfonate mixture.

We observed that the composition 7 according to the invention containing as a foaming surfactant only the sulfonate composition and the foaming booster has a foam density comparable to the foam density of the composition 8 (outside the invention) containing a sulfate surfactant and soaps.

1. Composition containing, in an aqueous medium, at least:
1) a sulfonate mixture containing:
a) an internal olefin sulfonate (A) having 16 carbon atoms and
b) an internal olefin sulfonate (B) having 18 carbon atoms,
wherein the weight ratio (A/B) of the component (A) to the component (B) present in the sulfonate mixture is from 75/25 to 90/10, preferably from 80/20 to 85/15, and
wherein the weight ratio of the hydroxy form compounds present in the internal olefin sulfonates (A) and (B) to the olefin form compounds present in the internal olefin sulfonates (A) and (B) is from 75/25 to 100/0, preferably from 80/20 to 95/5;
and 2) at least one foam-enhancing agent or foam booster.

2. Composition according to claim 1, wherein the sulfonate mixture comprises the internal olefin sulfonates (A) and (B) having a sulfonate group on the carbon atom located at C-2 position in a total amount of 28% by weight or less relative to the total weight of the sulfonate mixture, and preferably, from 10 to 28% by weight, and better from 15 to 25% by weight.

3. Composition containing, in an aqueous medium, at least:
1) a sulfonate mixture comprising:
one or more compounds of formula (A1): 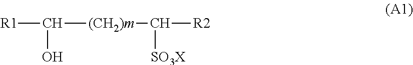
in which:
R1 and R2 represent, independently, a hydrogen atom or a branched or linear C₁-C₁₄ alkyl radical;
m is an integer from 0 to 14,
R1, R2 and m are such that the said compound(s) (A1) comprise 16 carbon atoms and
X represents a cation or a mixture of cations allowing achievement of electroneutrality of the said compound(s) (A1);
one or more compounds of formula (B1): 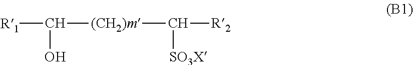
in which:
R′1 and R′2 represent, independently, a hydrogen atom or a branched or linear C₁-C₁₆ alkyl radical;
m′ is an integer from 0 to 16,
R′1, R′2 and m′ being such that the said compound(s) (B1) comprise 18 carbon atoms and
X′ represents a cation or a mixture of cations allowing achievement of electroneutrality of the said compound(s) (B1); and
optionally one or more compounds of formula (A2) and/or (A3): 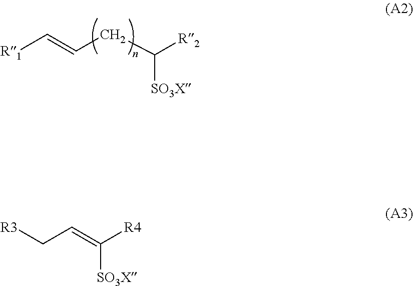
in which:
R″1, R″2, R3 and R4 represent, independently, a hydrogen atom or a branched or linear C₁-C₁₃ alkyl radical;
n is an integer from 0 to 13,
R″1, R″2, n, R3 and R4 being such that the said compound(s) (A2) and (A3) comprise 16 carbon atoms, and
X″ represents a cation or a mixture of cations allowing achievement of electroneutrality of the said compound(s) (A2) and/or (A3); and
optionally one or more compounds of formula (B2) and/or (B3): 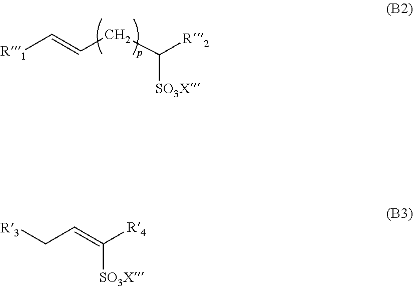
in which:
R′″1, R′″2, R′3 and R′4 represent, independently, a hydrogen atom or a branched or linear C₁-C₁₅ alkyl radical;
p is an integer from 0 to 15,
R′″1, R′″2, p, R′3 and R′4 being such that the said compound(s) (B2) and (B3) comprise 18 carbon atoms, and
X′″ represents a cation or a mixture of cations allowing achievement of electroneutrality of the said compound(s) (B2) and/or (B3);
wherein:
the weight ratio of all the compounds (A1), (A2) and (A3) to all the compounds (B1), (B2) and (B3) (which means (A1+A2+A3)/(B1+B2+B3)) is from 75/25 to 90/10, preferably from 80/20 to 85/15, and
the weight ratio of all the compounds (A1) and (B1) to all the compounds (A2), (B2), (A3) and (B3) (which means (A1+B1)/(A2+A3+B2+B3)) is from 75/25 to 100/0, preferably from 80/20 to 95/5;
2) at least one polymeric suspension agent.

4. Composition according to claim 3, wherein the sulfonate mixture comprises the compounds having a sulfonate group located on the carbon atom at C2 position, in particular the compounds (A1), (B1) and optionally the compounds (A2), (B2), (A3) and (B3) in a total amount of 28% by weight or less relative to the total weight of the sulfonate mixture, more preferably, from 10 to 28% by weight, and better from 15 to 25% by weight.

5. Composition according to claim 3 or 4, wherein the sulfonate mixture comprises
the compound(s) (A1) in an amount of from 50 to 90% by weight, preferably from 55 to 85% by weight, more preferably from 60 to 80% by weight, relative to the total weight of the sulfonate mixture and/or
the compound(s) (B1) in an amount of from 5 to 25% by weight, preferably from 10 to 23% by weight, more preferably from 15 to 20% by weight, relative to the total weight of the sulfonate mixture and/or
the compound(s) (A2) and (A3) in a total amount of from 0 to 20% by weight, preferably from 1 to 20% by weight, more preferably from 5 to 15% by weight, relative to the total weight of the sulfonate mixture and/or
the compound(s) (B2) and (B3) in a total amount of from 0 to 7% by weight, preferably from 0.5 to 5% by weight, more preferably from 1 to 4% by weight, relative to the total weight of the sulfonate mixture.

6. Composition according to any one of claims 1 to 5, containing an aqueous physiologically acceptable medium and preferably an aqueous cosmetically acceptable medium.

7. Composition according to any one of claims 1 to 6, wherein the sulfonate mixture is present in an amount from 0.5% to 20% by weight, more preferably from 1% to 15% by weight, more particularly from 1% to 10% by weight relative to the total weight of the composition.

8. Composition according to any one of claims 1 to 7, wherein the foam-enhancing agent or the foam booster is selected from:
the fatty alcohols;
the polyalkyleneglycols;
the polyglyceryl fatty acid esters;
the polyalkyleneglycol ethers of alkylglucose;
the celluloses;
the crosslinked polymers formed by the reaction of an C₁₀-C₁₈ alkylglucoside with 1,3-dichloro-2-propanol, and sulfonated with 3-chloro-2-hydroxypropyl sulfonate;
the fatty acid alkanolamides;
and their mixtures and more preferably selected from the polylethyleneglycols and polyglyceryl fatty acid esters and more particularity selected from PEG-45M and POLYGLYCERYL-2 LAURATE.

9. Composition according to any one of claims 1 to 8, wherein the foam-enhancing agent or the foam booster is present from 0.05% to 10% by weight, more preferably from 0.1% to 5% and much more preferably from 0.2% to 3% by weight by weight relative to the total weight of the composition.

10. Composition according to any one of claims 1 to 9, characterized in that it is sulfate surfactant-free.

11. Composition according to any one of claims 1 to 10, which further contains at least one fatty acid salt (soap), preferably chosen from potassium salts and sodium salts of C₁₀-C₂₄ fatty acids, preferably C₁₂-C₂₀ fatty acids, preferably C₁₂-C₁₈ fatty acids and more preferably the potassium salts of C₁₂-C₁₈ fatty acids, more particularity potassium salts of lauric acid, stearic acid, palmitic acid and their mixtures.

12. Composition according to any one of claims 1 to 11, which further contains at least one amphoteric or zwitterionic surfactant, preferably chosen from betaines, N-alkylamidobetaines and derivatives thereof, sultaines, alkyl polyaminocarboxylates, alkylamphoacetates, and mixtures thereof, and more preferably betaines and especially alkylbetaines and more particularity Coco betaine.

13. Composition according to any one of claims 1 to 12, which contains, in an aqueous medium:
a) at least one sulfonate mixture as defined in the preceding claims; and
b) at least one enhancing-foam agent or foam booster as defined in the preceding claims; and
c) at least one fatty acid salt as defined in the preceding claims; and
d) at least one amphoteric or zwitterionic surfactant as defined in the preceding claims.

14. Composition according claim 13, which contains, in an aqueous medium:
a) at least one sulfonate mixture as defined in the preceding claims from 0.5% to 20% by weight relative to the total weight, preferably from 1% to 15% by weight, more preferably from 1% to 10% by weight; and
b) at least one enhancing-foam agent or foam booster as defined in the preceding claims in an amount from 0.05% to 10% by weight relative to the total weight of the composition, preferably from 0.1% to 5% by weight, more preferably from 0.2% to 3% by weight; and
c) at least one fatty acid salt as defined in the preceding claims from 0.5% to 40% by weight relative to the total weight of the composition, preferably from 1% to 40% by weight, more preferably from 2% to 30% by weight; and
d) at least one amphoteric or zwitterionic surfactant as defined in the preceding claims in an amount from 0.1% to 20% by weight relative to the total weight of the composition, preferably from 0.15% to 15% by weight, more preferably from 1% to 10% by weight.

15. Composition according to any one of claims 1 to 14, which further contains at least one polymeric suspending agent, preferably selected from:
the water-soluble polysaccharides polymers and preferably selected from starches, carrageenan, xanthan gum, guar gum, and celluloses such as hydroxyethylcellulose and hydroxypropylcellulose, and mixtures thereof;
the associative or non-associative crosslinked homopolymers and crosslinked copolymers of acrylic acid and/or methacrylic acid and/or their esters;
the non-ionic associative polyether-urethanes.

16. Composition according to any one of claims 1 to 15, wherein the pH thereof is from 7.0 to 10 and more preferably from 8 to 9.

17. Process for cleansing keratin materials, which consists of applying to the said keratin materials a composition according to any one of claims 1 to 16, in working the said composition into a foam and then rinsing off the said composition, especially with water.
